(19) United States
(12) Patent Application Publication
(10) Pub. No.: US 2025/0154276 A1
(43) Pub. Date: May 15, 2025

(54) APPLICATION OF CD45 AS BIOMARKER IN SCREENING EFFECTIVENESS AND PRECISION OF CD26 ANTIBODY OR DERIVATIVE THEREOF IN TREATING TUMORS

(71) Applicant: ZONHON BIOPHARMA INSTITUTE, INC., Changzhou, Jiangsu (CN)

(72) Inventors: Bruce Yong MA, Changzhou, Jiangsu (CN); Peipei CAO, Changzhou, Jiangsu (CN); Chennan GE, Changzhou, Jiangsu (CN); Jianhua HANG, Changzhou, Jiangsu (CN)

(21) Appl. No.: 18/834,319

(22) PCT Filed: Jan. 17, 2023
(86) PCT No.: PCT/CN2023/072600

§ 371 Date: Jul. 30, 2024

(30) Foreign Application Priority Data
Jan. 30, 2022 (CN) 202210113797.0

(51) Int. Cl. C07K 16/28; A61K 39/00; A61K 40/11; A61K 40/31; A61P 35/00; C12N 15/86; G01N 33/50

(52) U.S. Cl. CPC C07K 16/2896; A61K 40/11; A61K 40/31; A61P 35/00; C12N 15/86; G01N 33/5011; A61K 2039/505; A61K 2239/13; C12N 2750/14143; G01N 2333/70589; G01N 2800/52

(57) ABSTRACT

CD45 is used as a biomarker in screening the effectiveness of a CD26 antibody or derivative thereof in treating tumors in a test subject. Specifically, when CD45 is not expressed or has low expression, determining that the treatment of the test subject who has received a CD26 antibody or a derivative thereof is effective or has a high probability of being effective; and when CD45 expression is high or relatively high, determining that the treatment of the test subject who has received a CD26 antibody or a derivative thereof is ineffective or has a high probability of being ineffective. It has been found that CD26 acts as a new mechanism for treatment effectiveness and precision of novel tumor immunotherapy targets.

### TECHNICAL FIELD

The invention relates to a biomarker predicting clinical effectiveness, including the use of CD45 as a biomarker predicting clinical effectiveness, more specifically including CD45 as a biomarker in screening effectiveness and precision of CD26 antibody or derivative thereof in treating tumors.

### BACKGROUND OF THE INVENTION

Precision medicine is to customize the diagnosis, treatment and prognosis of different patients based on clinical pathological and molecular characteristics, including precision prevention (risk prediction and preventive intervention), precision diagnosis (early detection and diagnosis of disease, molecular typing), precision treatment (molecular target therapy, prediction and monitoring of efficacy, precision surgical techniques, etc.).

The discovery and use of biomarkers is the key research direction of precision medicine. According to different uses, biomarkers can be divided into: diagnostic biomarkers to define disease status or classification, prognostic biomarkers to reflect disease prognosis, predictive biomarkers to predict response to an intervention, pharmacodynamic biomarkers to reflect biologic response to treatment, and safety biomarkers to avoid or mitigate safety risks. Predictive biomarkers can be used to screen patients who are more likely to benefit from treatment and exclude patients who have no clinical benefit. Immune checkpoint inhibitors such as PD-1/PD-L1 and CTLA-4 have been widely studied in a variety of solid tumors, and have been used as the first-line treatment for non-small cell lung cancer. However, the overall objective response rate is still only about 20%. PD-L1 expression can be used as a predictive marker of response to anti-PD-1/PD-L1 treatment, and tumor mutation burden (TMB) has also been shown to be associated with the efficacy of immune checkpoint inhibitors in melanoma, lung adenocarcinoma, and bladder cancer.

CD26 is a multifunctional type II transmembrane glycoprotein that can also be found in plasma in a dissolved form. CD26 usually exists as a homodimer with a monomer comprising 766 amino acids and a relative molecular mass of about 110 kDa. The amino acid residues of CD26 can be divided into five parts from inside to outside: intracellular region (1-6), transmembrane region (7-28), highly glycosylated region (29-323), cysteine-rich region (324-551) and C-terminal catalytic domain (552-766). The molecular structure of CD26 is closely related to its function. CD26 (DPP4) inhibitors have been used in the treatment of type 2 diabetes for decades. The expression of CD26 is significantly increased on the surface of a variety of tumor cells, such as malignant mesothelioma, renal cancer, prostate cancer, lung cancer, etc. CD26 is a valuable target for these types of tumors with high CD26 expression (CD26 is a potential biomarker and target for cancer therapy Pharmacology& Therapeutics (2019), 198:135-159).

At present, the most advanced anticancer drug research targeting human CD26 is the monoclonal antibody YS110 from Y's Therapeutics, which has completed phase I/II clinical trials. However, according to the existing literature, YS110 has been found to have low activity in preclinical studies both in vitro and in vivo. As in the literature “A humanized anti-CD26 monoclonal antibody inhibits cell growth of malignant mesothelioma via retarded G2/M cells In cycle transition, Cancer Cell Int (2016) 16:35”, after treated with 250 ug/ml YS110 for 48 h, the growth inhibition rate of tumor cell line was 18.3%, and the IC₅₀ value was much higher than 250 ug/ml. These results indicated that its activity in vitro was low. In the patent “anti-CD26 antibody and use thereof” (Application No. CN200680034937.4), after the treatment of YS110 in mice bearing a variety of CD26 high expression tumor cell lines, YS110 can only reduce tumor volume and alleviate tumor growth to a certain extent, but can not completely inhibit tumor growth to achieve tumor cure, showing its activity in animal models is still not ideal. Results from the completed phase 2 clinical study of YS110 have shown that YS110 is well tolerated, but disease control rates have not been as high as expected, which corresponds to the low activity of YS110 in preclinical studies.

In the CD26 antibody research, the inventor found that some CD26+ cells were sensitive to CD26 antibodies or derivative thereof, but some CD26+ cells is not sensitive to CD26 antibody. Combined with the low in vitro and in vivo activity of YS110 in preclinical and clinical studies, the inventors speculates that the CD26 antibody acted selectively on CD26+ cells. To find biomarkers that can effectively predict response to anti-CD26 therapy, is an urgent problem to be solved for the medical use of anti-CD26 antibody.

### SUMMARY OF THE INVENTION

This application provides CD45 as a biomarker screening the effectiveness of the treatment with CD26 antibodies or derivative thereof.

Firstly, this application provides the use of CD26 antibodies or derivative thereof in the manufacture of a medicament for treating a tumor, the tumor expresses CD26 but without CD45 expression. The tumor comprises solid tumor and hematological tumor.

The present application also provides the use of the CD26 antibody or derivative thereof in the manufacture of a medicament for treating a tumor, the tumor expresses CD26 and with a low CD45 expression. The tumor comprises solid tumor and hematological tumor.

The “antibody” can be either a full-length antibody containing two heavy chains and two light chains; It can also be an antigen-binding fragment, that is, an antibody fragment that retains the ability to specifically bind to an antigen, such as a fragment that retains one or more CDR regions, including but not limited to Fab fragment, FV fragment, linear antibody, single chain antibody fragment, nanoantibody, bispecific antibody, and multi-specific antibody.

The “derivative” means that the active drug molecule is not traditional antibody (full-length antibody or antigen-binding fragment), but other forms of antibody, including but not limited to CAR-T targeting CD26, etc., or other forms which can generate antibodies in the body, including but not limited to rAAV vector (adeno-associated viral vector) containing anti-CD26 antibody coding gene.

Secondly, the application provides the use of CD45 antigen or anti-CD45 antibody in the preparation of reagent for screening the effectiveness of CD26 antibody or derivative thereof in tumor treatment.

The above reagent is used as follows: detecting CD45 expression in the lesions; when CD45 has a low expression, it is determined that the treatment with CD26 antibody or derivative thereof is effective or has a high probability of effectiveness; when CD45 has a high expression, it is determined that the treatment with CD26 antibody or derivative thereof is ineffective or has a high probability of ineffectiveness.

Alternatively, the reagent is used as follows: detecting CD45 expression in the lesions; when CD45 has no expression, it is determined that the treatment with CD26 antibody or derivative thereof is effective or has a high probability of effectiveness; when CD45 has an expression, it is determined that the treatment with CD26 antibody or derivative thereof is ineffective or has a high probability of ineffectiveness.

Thirdly, the application provides a method for screening the effectiveness of treatment with CD26 antibody or derivative thereof: detecting CD45 expression in lesions; when CD45 has a low expression, it is determined that the treatment with CD26 antibody or derivative thereof to the subject is effective or has a high probability of effectiveness; when CD45 has a high expression, it is determined that the treatment with CD26 antibody or derivative thereof to the subject is ineffective or has a high probability of ineffectiveness.

Optimally, the application provides a method for screening effective treatment with CD26 antibody or derivative thereof: detecting CD45 expression in lesions; when CD45 has no expression, it is determined that the treatment with CD26 antibody or derivative thereof to the subject is effective or has a high probability of effectiveness; when CD45 has an expression, it is determined that the treatment with CD26 antibody or derivative thereof to the subject is ineffective or has a high probability of ineffectiveness.

CD45 is a transmembrane protein tyrosine phosphatase (PTPase), which is widely expressed in blood cells. It is composed of extracellular domain, transmembrane domain and intracellular domain. There are a variety of CD45 subtypes. Different subtypes have different extracellular domains and have the same intracellular and transmembrane domains. The extracellular domain of CD45 is a fragment of 391-552 amino acids, with 11-15 N-glycosylation sites and multiple O-glycosylation sites. The intracellular domain of CD45 is highly conserved and contains two repeat protein tyrosine phosphatase (PTPase) domains, one of which has PTPase activity, and the other has no significant PTPase activity due to the key amino acid change, and the latter may regulate the PTPase activity of the former. CD45 plays a key role in lymphocyte development and activation by regulating Src family protein kinases through its PTPase activity in the cytoplasmic region. Whether CD45 is involved in the pharmacologic effect of the CD26 antibody is unknown prior to this application.

This application provides the use of CD45 in screening the effectiveness of CD26 antibody or derivative thereof, which can effectively improve the accuracy and effectiveness of CD26 antibody or derivative thereof in tumor immunotherapy, and improve the clinical benefit of patients. Novel mechanism for the effectiveness and accuracy of CD26 therapy as new target for cancer immunotherapy has been identified in this application.

### BRIEF DESCRIPTION OF THE DRAWINGS

FIG. 1: Detection of CD45, CD4, and CD26 expression of CD4+T cells by flow cytometry

FIG. 2: Detection of CD45, CD8, and CD26 expression of CD8+T cells by flow cytometry

FIG. 3: Antibody-mediated killing effect of PBMC on cells

FIG. 4: Tumor volume in NOD-SCID mice subcutaneously bearing PC-3 cell with the administration of 18G272 or 19G294

FIG. 5: Body weight of NOD-SCID mice subcutaneously bearing PC-3 cell with the administration 18G272 or 19G294

FIG. 6: Tumor volume in NOD-SCID mice subcutaneously bearing NCI-H596 cell with the administration of 18G272

FIG. 7: Tumor volume in NOD-SCID mice subcutaneously bearing NCI-H226 cell with the administration of 19G294

FIG. 8: Body weight of NOD-SCID mice subcutaneously bearing NCI-H226 cell with the administration of 19G294

FIG. 9: Tumor volume in NOD-SCID mice subcutaneously bearing A498 cell with the administration of 19G294

FIG. 10: Tumor volume in NOD-SCID mice subcutaneously bearing OS-RC-2 cells with the administration of 18G272

FIG. 11: Tumor volume in NOD-SCID mice subcutaneously bearing OS-RC-2 cells with the administration of 19G294

FIG. 12: Diagram of the AAV expression backbone vector, including pUC ori, Amp resistance gene, 5′ ITR of adeno-associated virus, CMV promoter, β intron-enhancer sequence, multiple cloning site, poly A termination sequence and 3′ ITR.

FIG. 13: Tumor volume changing in NOD/SCID mice bearing OS-RC-2 cells after delivering BiTE gene by AAV.

FIG. 14: Body weight changing in NOD/SCID mice bearing OS-RC-2 cells with the administration of AAV-delivering BiTE gene

FIG. 15: Survival of NOD/SCID mice bearing OSRC-2 with the administration after delivering BiTE gene by AAV.

FIG. 16: Detection of CAR-positive rate in CAR-T cells by flow cytometry

FIG. 17: Killing effect of CD26-targeting CAR-T cells on tumor cells

### DETAILED DESCRIPTION OF THE INVENTION

Unless clearly defined, technical terms used herein have meanings generally understood by one of ordinary skill in the art.

Singular words such as “a (a, an)” and “the (the)” include their corresponding plural form.

The term “or” means “and/or” and can be used interchangeably with “and/or”.

The term “CD26” is also known as dipeptidyl peptidase 4 (DPP4). The amino acid sequence of human CD26 can be found in Genbank with accession number NP_001926.2, and its cDNA sequence can be found in Genbank with accession number NM_001933.5.

The term “antibody” refers to a family of immunoglobulins that can specifically bind the corresponding antigen noncovalently and reversibly. For example, naturally IgG antibodies are tetamers that contain at least two heavy chains and two light chains linked to each other by disulfide bonds. Each heavy chain is composed of a heavy chain variable region (VH) and a heavy chain constant region. The heavy chain constant region consists of three domains CH1, CH2, and CH3. Each light chain consists of a light chain variable region (VL) and a light chain constant region. The light chain constant region consists of one domain, CL. VH and VL can be further subdivided into complementarity determining regions (CDR, also known as hypervariable regions) with high variability, and a more conserved framework region (FR). Each VH and VL consists of three CDRs and four FRs, arranged in the following order from amino-terminal to carboxyl terminal: FR1, CDR1, FR2, CDR2, FR3, CDR3, and FR4. HCDR1, HCDR2 and HCDR3 are three complementarity determining regions of heavy chain, and LCDR1, LCDR2 and LCDR3 are three complementarity determining regions of light chain. The heavy chain variable region (VH) and light chain variable region (VL) are responsible for antigen recognition, especially complementarity-determining regions (CDR), which are usually specific for different epitopes of antigens. The constant region is primarily responsible for effector functions.

The term “BiTE” is a bispecific antibody, full name “bispecific T cell engager”, formed by connecting two single chain antibody fragments (scFv) through a linker. One scFv targets tumor-associated antigens, and another scFv targets CD3 on T cell. The single chain antibody fragment (scFv) is formed by connecting heavy chain variable region and light chain variable region through a linker.

CD3 is a component of T-cell signaling. When BiTE binds to both T cells and tumor cells, T cells are activated, which promotes CD8+T cells to directly secrete perforin and granzyme, and promotes CD4+T cells to secrete cytokines to recruit and activate killer T cells, thereby killing tumor cells.

In specific embodiments, “bispecific antibodies” such as 18G272 and 19G294 are both BiTE targeting CD26 (ZHBITE), with sequences as shown in SEQ ID NO: 1 and SEQ ID NO: 2, respectively.

ZB: ZHBody, with BiTE molecule fused to Fc segment, from the N-terminus to the C-terminus include: BiTE-linker-CH2-CH3-linker-CH2-CH3. The amino acid sequence of ZB09 is shown as SEQ ID NO: 4. ZB antibody can be prepared according to conventional methods, such as the codon optimization of the antibody sequence, adding AvrII restriction enzyme recognizing sequence and kozak sequence upstream of the gene, and adding termination codon and BstZ17I restriction enzyme recognizing sequence downstream of the gene. After whole gene synthesis, the target gene is obtained by PCR amplification. The target gene is inserted between the AvrII and BstZ17I sites of the pCHO1.0 vector by enzyme digestion and ligation to generate the expressing vector. The expressing vector is stably transformed into CHO-S cells, and the stable cell lines with high expression are screened after MTX and puromycin treatment. The high expression stable cell lines are cultured in Dynamis medium (A2617501, Thermo Fisher) at 37° C., 8% CO₂, 130 rpm for fed-batch culture. The supernatant of cultured medium is centrifuged, and the supernatant is collected, filtered through 0.45 μm filter membrane, and purified by chromatography. The target antibody molecule is obtained.

ZA: ZHBiAb, bispecific IgG full-length antibody, the structure is basically the same as natural antibody, with two heavy chains and two light chains, each of the light chain is connected to a heavy chain through disulfide bond. The two light-heavy chain dimers are linked by a disulfide bond between the heavy chains, resulting in the formation of a Y-shaped molecule. One of the Fab fragments in ZA16 and ZA23 recognizes CD26 and the other recognizes CD3. In ZA16, amino acid sequence of heavy chain A is shown as SEQ ID NO: 5, amino acid sequence of light chain A is shown as SEQ ID NO: 6, amino acid sequence of heavy chain B is shown as SEQ ID NO: 7, amino acid sequence of light chain B is shown as SEQ ID NO: 8. In ZA23, amino acid sequence of heavy chain A is shown as SEQ ID NO: 9, amino acid sequence of light chain A is shown as SEQ ID NO: 10, amino acid sequence of heavy chain B is shown as SEQ ID NO: 11 and amino acid sequence of light chain B is shown as SEQ ID NO: 12.

CD26-targeting CAR-T cells are T cells that express CD26 CAR molecules. The molecular structure of CD26 CAR in this application includes CD8a signal peptide, anti-CD26 single chain antibody fragment shown as VL-Linker-VH, CD8a hinge, CD8a transmembrane domain, CD28 costimulatory domain, and CD3zeta intracellular domain.

The rAAV vector containing the gene coding anti-CD26 antibody is a recombinant adeno-associated virus (rAAV) vector to deliver the anti-CD26 antibody gene to the body. The rAAV-infected cells continuously synthesize anti-CD26 antibodies.

### Example 1 Effect of CD26 Antibody on PBMC Cells

Peripheral blood mononuclear cell (PBMC) is mainly composed of T lymphocytes, B lymphocytes, monocytes, macrophages, dendritic cells and other immune cells, and they all express CD26. To observe whether CD26 antibodies have a killing effect on normal PBMC cell and the correlation with CD45 expression, the following tests were carried out:

### 1. CD26, CD45 Positive Rate Detection of Cell Lines

The adherent cells were cultured in T75 cell culture flask. When the cell fusion was more than 80%, the cells were digested with trypsin and collected, washed once with MACS buffer, and counted with a hemocytometer. The cells were divided into 5×10⁵ cells each. Anti-CD26 monoclonal antibody used as the primary antibody were incubated with the target cells at room temperature for about 40 min. After incubation, the product was centrifuged, and the supernatant was discarded. The cell precipitates were resuspended in MACS buffer, the product was centrifuged, and the supernatant was discarded and the cell precipitates were collected. Then Alexa Fluor 488 mouse anti-human IgG1 was used as the secondary antibody. The cell precipitates were resuspended and incubated at room temperature in a dark place for about 30 min. After incubation, the cells were washed twice with PBS, the product was centrifuged and the supernatant was discarded, the cell precipitates were collected. The cell precipitate was resuspended in approximately 200 ul of MACS buffer. Within 1 h, the CD26 positive rate was detected and analyzed with a flow cytometer ACCURI C6, and the results were shown in Table 1.

The cells were cultured in T75 cell culture flask. Then the cells were collected and washed once with MACS buffer, and counted with a hemocytometer. The cells were divided into 5×10⁵ cells each. Anti-CD45-FITC monoclonal antibody (article number 130-113-679, Miltenyi, can recognize all CD45 subtypes) were incubated with the target cells at 2-8° C. for about 10 min. After incubation, the product was washed twice with MACS buffer and centrifuged, the supernatant was discarded. The cell precipitates were resuspended in 200 ul of MACS buffer. Within 1 h, the CD45 positive rate was detected and analyzed with a flow cytometer ACCURI C6, and the results were shown in Table 1.

TABLE 1

| Cells | CD26 | CD45 |
|---|---|---|
| PBMC | 75% | 99.2% |

### 2. CD26 Antibody Mediated Toxicity of PBMC to PBMC

### 1). Experimental Methods

Human renal cancer cell 786-0 and OS-RC-2 models were prepared as described below.

### 786-0 Cell Model

PBMC cells were labeled with green fluorescent signal by fluorescent dye Calcein-AM. 786-0 cells were seeded into U-bottom 96 well plate at a cell concentration of 6×10⁵ cells/ml, 50 μl per well. 50 μl antibody with final concentration of 100 ng/ml, 10 ng/ml, 1 ng/ml, 0.1 ng/ml, 0.01 ng/ml, 0.001 ng/ml were added to the corresponding well. Add 50 μl medium to the blank control well, add 50 μl 1% TritonX-100 to the positive control well. Then 50 μl PBMC cells with concentration of 9×10⁶ cells/ml were added at the E/T ratio of 10:1. The reaction system was incubated at 37° C. in carbon dioxide for 5 h. After the reaction and centrifugation, the supernatant was placed in a new 96-well plate. After centrifugation again, 80 μl of the supernatant was placed in a black 96-well plate and detected using a microplate reader at 470 nm excitation light wavelength and 515 nm emission wavelength.

### OS-RC-2 Cell Model

PBMC cells were labeled with green fluorescent signal by fluorescent dye Calcein-AM. OS-RC-2 cells were seeded into U-bottom 96 well plate at a cell concentration of 6×10⁵ cells/ml, 50 μl per well. 50 μl antibody with final concentration of 100 ng/ml, 10 ng/ml, 1 ng/ml, 0.1 ng/ml, 0.01 ng/ml and 0.001 ng/ml were added to the corresponding wells. 50 μl of culture medium was added to the blank control wells. 50 μl 1% TritonX-100 was added to the positive control well. Then 50 μl PBMC cells with concentration of 9×10⁶ cells/ml were added at the E/T ratio of 15:1. The reaction system was incubated at 37° C. in carbon dioxide for 5 h. After the reaction and centrifugation, the supernatant was placed in a new 96-well plate. After centrifugation again, 80 μl of the supernatant was placed in a black 96-well plate and detected using a microplate reader at 470 nm excitation light wavelength and 515 nm emission wavelength.

Cell lysis rate=(Vₛₐₘₚₗₑ−V_{vehicle control})/(V_{TritonX-100}−V_{vehicle control})×100%. Vₛₐₘₚₗₑ is the mean value of the fluorescence signal reading of the drug treatment group, V_{vehicle control} is the mean value of the fluorescence signal reading of the blank control group, and V_{Triton-100} is the mean value of the fluorescence signal reading of the positive control group. The IC₅₀ values of each sample mediating toxicity of PBMC to PBMC were calculated based on cell lysis rate and sample concentration by GraphPad Prism 7.00 software.

### 2). Results Analysis

In the process of PBMC killing 786-0 mediated by antibody 18G272, 18G272 did not mediate killing effect of PBMC to PBMC cells. The IC₅₀ value of 18G272 mediating cytotoxicity of PBMC (4 #) to PBMC (4 #) cells was 99730000 pg/ml. PBMC (6 #) had no 18G272-mediated cytotoxic effect on PBMC (6 #) cells.

In the process of PBMC killing OS-RC-2 mediated by antibody 18G272, 18G272 did not mediate killing effect of PBMC to PBMC cells. PBMC (1 #) had no 18G272-mediated cytotoxic effect on PBMC (1 #) cells. PBMC (3 #) had no 18G272-mediated cytotoxic effect on PBMC (3 #) cells.

In the process of PBMC killing 786-0 mediated by antibody 19G294, 19G294 did not mediate killing effect of PBMC to PBMC cells. The IC₅₀ value of 19G294 mediating cytotoxicity of PBMC (2 #) to PBMC (2 #) cells was 1915313 pg/ml. The IC₅₀ value of 19G294 mediating cytotoxicity of PBMC (3 #) to PBMC (3 #) cells was 1947224 pg/ml.

In the process of PBMC killing OS-RC-2 mediated by antibody 19G294, 19G294 did not mediate killing effect of PBMC to PBMC cells. The IC₅₀ value of 19G294 mediating cytotoxicity of PBMC (2 #) to PBMC (2 #) cells was 47920315 pg/ml. The IC₅₀ value of 19G294 mediating cytotoxicity of PBMC (3 #) to PBMC (3 #) cells was 1406115138 pg/ml.

In conclusion, PBMC expressed CD26 and highly expressed CD45, and antibodies targeting CD26 did not mediate cytotoxicity of PBMC to PBMC.

### 2. CD26 Antibody Mediated Toxicity of PBMC to CD4+ T Cells and CD8+ T Cell

### Experiment 1: CD4+T and CD8+T Cells were Isolated from PBMC

### 1). Methods

CD4+ T cells were isolated from PBMC cell using CD4+ magnetic beads (130-045-101, Miltenyi), CD8+ T cells were isolated from PBMC cell using CD8 magnetic beads (130-045-201, Miltenyi).

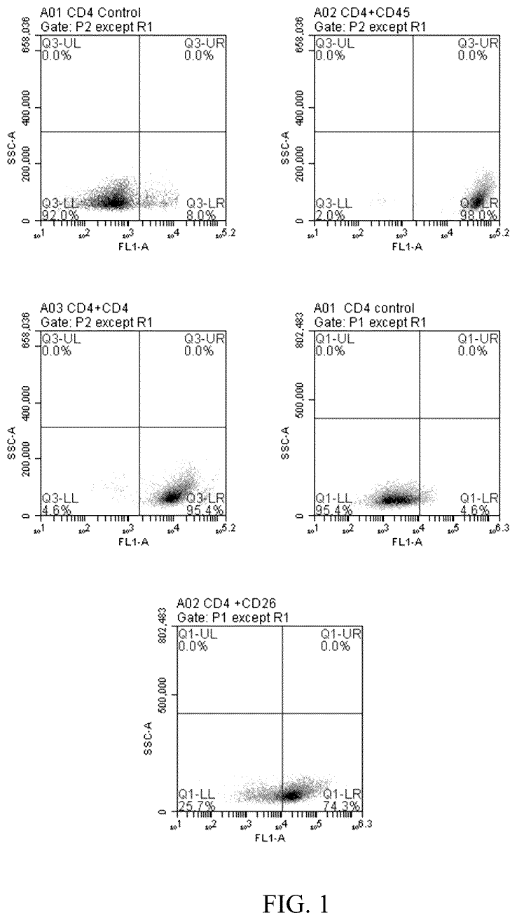

The isolated CD4+T cells were washed once with MACS buffer and counted by a hemacytometer. The cells were divided into 5×10⁵ cells each. Anti-CD4 antibody (product No. 53-0048-42, e Bioscinece) and anti-CD45-FITC monoclonal antibody (product No. 130-113-679, Miltenyi, can recognize all CD45 subtypes) were diluted according to the specification, incubated with target cells at 2-8° C. for about 10 minutes, washed twice with MACS buffer and centrifugated. The precipitate was collected and resuspended with about 200 μl MACS buffer, detected with a flow cytometer ACCURI C6 in 1 hour, and the results were shown in FIG. 1 and Table 2.

TABLE 2

| Cells | CD4 | CD45 | CD26 |
|---|---|---|---|
| CD4+ T cell | 95.4% | 98% | 74.3% |

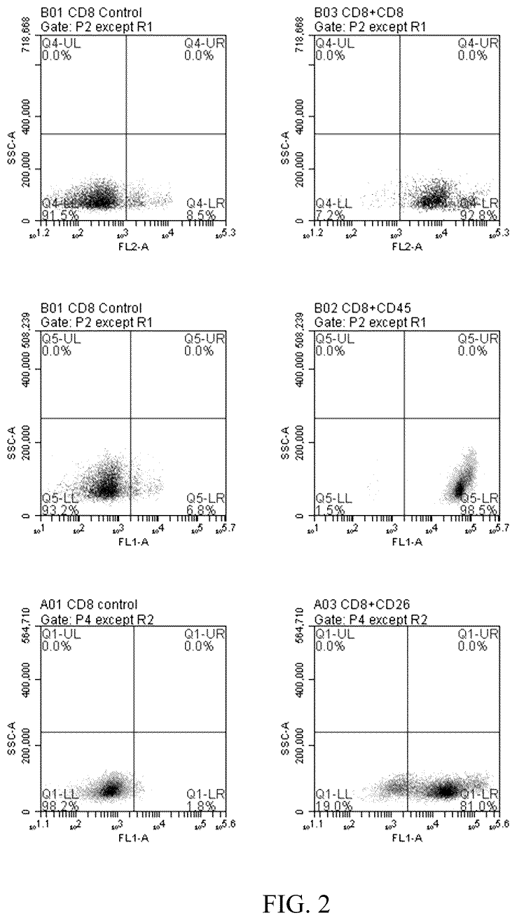

The isolated CD8+ T cells were washed once with MACS buffer and counted by a hemacytometer. The cells were divided into 5×10⁵ cells each. Anti CD8 antibodies (130-098-075, Miltenyi) and antiCD45-FITC monoclonal antibody (130-113-679, Miltenyi, can recognize all CD45 subtypes) were diluted according to the specification, incubated with target cells at 2-8° C. for 10 min, washed twice with MACS buffer and centrifugated. The precipitate was collected and resuspended with about 200 μl MACS buffer, detected with a flow cytometer ACCURI C6 in 1 hour, and the results were shown in FIG. 2 and Table 3.

TABLE 3

| Cells | CD8 | CD45 | CD26 |
|---|---|---|---|
| CD8+ T cell | 92.8% | 98.5% | 81.0% |

### Experiment 2: Cytotoxicity Test

### 1). Methods

CD4+ T and CD8+ T cells were labeled with green fluorescent signal by fluorescent dye Calcein-AM. The cells were seeded into U-bottom 96 well plate at a cell concentration of 3×10⁵ cells/ml, 50 μl per well. 50 μl of antibody with final concentration of 100 ng/ml, 10 ng/ml, 1 ng/ml, 0.1 ng/ml, 0.01 ng/ml and 0.001 ng/ml were added to the corresponding wells. 50 μl of culture medium was added to the blank control well. 50 μl TritonX-100 (final concentration of 1%) was added to the positive control well. Then 50 μl PBMC cells with concentration of 4.5×10⁶ cells/ml were added at the E/T ratio of 15:1. The reaction system was incubated at 37° C. in carbon dioxide for 5 h and then centrifugated, the supernatant was placed in a new 96-well plate. After centrifugation again, 80 μl of the supernatant was placed in a black 96-well plate and detected with a microplate reader at 470 nm excitation light wavelength and 515 nm emission wavelength.

Cell lysis rate=(Vₛₐₘₚₗₑ−V_{vehicle control})/(V_{TritonX-100}−V_{vehicle control})×100%. Vₛₐₘₚₗₑ was the mean value of the fluorescence signal reading of the drug treatment group, V_{vehicle control} was the mean value of the fluorescence signal reading of the solvent control group, and V_{Triton-100} was the mean value of the fluorescence signal reading of the positive control group. The IC₅₀ values of each sample mediating toxicity of PBMC to PBMC were calculated based on cell lysis rate and sample concentration by GraphPad Prism 7.00 software.

### 2). Results Analysis

The results were shown in Table 4.

TABLE 4
Cytotoxicity of PBMC on CD4+ and CD8+T cells
mediated by anti-CD26 antibodies

| Cells | 19G294 |
|---|---|
| CD4+ T cell | NA* |
| CD8+ T cell | NA |

*NA indicated no detectable cytotoxic effect of PBMC on cells mediated by antibodies
According to the above results, CD4+T cells and CD8+T cells highly expressed CD45, and the antibody 19G294 targeting CD26 did not mediate the cytotoxic effect of PBMC cells on CD4+ and CD8+T cell.

### Example 2 Effect of CD26 Antibody on Blood Tumor

To observe whether CD26 antibody has a killing effect on blood tumor cells and the association with CD45 expression, the following experiments were performed:

### I. CD26, CD45 Positive Rate Detection in Lymphoma Cell Lines

The detection method was the same as Example 1, and the expression of CD26 and CD45 of different lymphoma cells was shown in Table 5.

TABLE 5

| Cells | CD26 | CD45 |
|---|---|---|
| U937 | 97.9% | 98.2% |
| Jurkat | 94.8% | 99.9% |
| HL-60 | 98.2% | 97.6% |
| SNK-6 | 99.4% | 94.5% |
| NAMALWA | 98.6% | 95.1% |
| MOLT-4 | 72.1% | 91.3% |
| Z-138 | 98.8% | 93.7% |
| Raji | No expression | — |

### II. Toxicity Evaluation of CD26 Antibody-Mediated PBMC to Lymphoma Cells

### 1. Methods

According to the following method, Jurkat cell lymphoma cells, U937 cells, Z-138, NAMALWA cell, the HL 60 cells, SNK, MOLT-4-6 cells models were prepared respectively.

The tumor cells were labeled with green fluorescent signal by fluorescent dye Calcein-AM. The cells were seeded into U-bottom 96 well plate at a cell concentration of 6×10⁵ cells/ml, 50 μl per well. 50 μl of antibody with final concentration of 100 ng/ml, 10 ng/ml, 1 ng/ml, 0.1 ng/ml, 0.01 ng/ml and 0.001 ng/ml were added to the corresponding wells. 50 μl of culture medium was added to the blank control well. 50 μl TritonX-100 (final concentration of 1%) was added to the positive control well. Then 50 μl PBMC cells with concentration of 9×10⁶ cells/ml were added at the E/T ratio of 15:1. The reaction system was incubated at 37° C. in carbon dioxide for 5 h. After the reaction and centrifugation, the supernatant was placed in a new 96-well plate. After centrifugation again, 80 μl of the supernatant was placed in a black 96-well plate and detected using a microplate reader at 470 nm excitation light wavelength and 515 nm emission wavelength.

Cell lysis rate=(Vₛₐₘₚₗₑ−V_{vehicle control})/(V_{TritonX-100}−V_{vehicle control})×100%. Vₛₐₘₚₗₑ is the mean value of the fluorescence signal reading of the drug treatment group, V_{vehicle control} is the mean value of the fluorescence signal reading of the solvent control group, and V_{Triton-100} is the mean value of the fluorescence signal reading of the positive control group. The IC₅₀ values of each sample mediating toxicity of PBMC to tumor cells were calculated based on cell lysis rate and sample concentration by GraphPad Prism 7.00 software

### 3. Results Analysis

Different types of bispecific antibodies ZA23, ZA16, ZB09, 19G294, etc did not mediate PBMC cytotoxicity to CD45+CD26+ tumor cell lines such as Jurkat, U937, Z-138, NAMALWA, HL-60, SNK-6, MOLT-4, etc. The results were shown in table 6, and representative figures were shown in FIG. 3.

TABLE 6
IC50 of different bispecific antibodies to tumor cells

| Cells | ZA23 | ZA16 | ZB09 | 19G294 |
|---|---|---|---|---|
| Jurkat | NA* | NA | NA | NA |
| U937 | NA | NA | NA | NA |
| Z-138 | NA | NA | NA | NA |
| SNK-6 | NA | NA | NA | NA |
| MOLT-4 | NA | NA | NA | NA |
| HL-60 | NA | NA | NA | NA |
| NAMALWA | NA | NA | NA | NA |

*NA indicated no detectable cytotoxic effect of PBMC on cells mediated by antibodies
In conclusion, the fluorescence signal in the figure did not change with the increase of antibody concentration (there was no dose-dependent relationship with antibody concentration), indicating that the antibody did not mediate the cytotoxic effect of PBMC cells on the corresponding cells. Most lymphoma cells express CD26 and highly express CD45. CD26 antibodies did not mediate cytotoxic effect of PBMC on lymphoma cells highly expressing CD45.

### Example 3 Effect of CD26 Antibody on Solid Tumors

To observe whether CD26 antibody has a killing effect on the solid tumor cells and the association with CD45 expression, the following experiments were performed:

### I. CD26 and CD45 Positive Rate Detection in Solid Tumor Cell Lines

The detection method was the same as example 1, and the expression of CD26 and CD45 of different solid tumor cells was shown in Table 7.

TABLE 7

| Cells | Tumors | CD26 | CD45 |
|---|---|---|---|
| OS-RC-2 | Renal cancer | 97.8% | No expression |
| A498 | | 97.1% | No expression |
| 786-0 | | 99.9% | No expression |
| G401 | | No expression | — |
| NCI-H596 | Lung cancer | 72.1% | No expression |
| NCI-H226 | lung squamous | 99.4% | No expression |
| | carcinoma | | |
| PC-3 | Prostate Cancer | 93.8% | No expression |

Note:
“—” indicated no detection; “No expression” indicated no expression was detected when using method of this application, which can be considered as no expression or low expression, but not strictly no expression at all, which may be related to the detection limit of the method.

### II. Toxicity Evaluation of CD26 Antibody-Mediated PBMC to Solid Tumor Cells

### 1. Methods

The method was the same as Example 2, prepare kidney cancer OS-RC-2 model, 786-0 model, human lung squamous carcinoma NCI-H226 model, prostate cancer PC-3 model.

### 2. Results Analysis

With the same experimental conditions, the bispecific antibodies ZA23, ZA16, ZB09, 19G294 mediated the cytotoxic effect of PBMC cells on CD26+ CD45− tumor cell OS-RC-2, as shown in table 8. Bispecific antibodies such as 19G294 and 18G272 can mediate strong cytotoxic effects of PBMC cells on a variety of CD26+CD45− tumor cells, as shown in Table 9.

TABLE 8
IC50 values (pM) of different bispecific antibodies
mediating PBMC cytotoxicity to OS-RC-2

| Cells | ZA23 | ZA16 | ZB09 | 19G294 |
|---|---|---|---|---|
| OS-RC-2 | 87.5 | 5.8 | 10.1 | 9.9 |

TABLE 9
IC50 values (pM) of different bispecific antibodies
mediating PBMC cytotoxicity to different target cells

| Cells | 18G272 | 19G294 |
|---|---|---|
| 786-0 | 41.4 | 4.7 |
| OS-RC-2 | 5.2 | 9.9 |
| NCI-H226 | 2.6 | 13.7 |
| PC-3 | 6.9 | 4.0 |

In conclusion, different CD26 antibodies killed a variety of solid tumor cell lines with no or low CD45 expression in vitro. In order to further observe in vivo efficacy of CD26 antibody on the solid tumor, carry out the following III-V in vivo experiment.

### III. Efficacy of Humanized Bispecific Antibodies 18G272 and 19G294 in NOD/SCID Mice Subcutaneously Bearing PC-3 Human Prostate Cancer Cell Xenografts

Methods: 5×10⁶ cells/0.1 ml PC-3 cell suspension and 1×10⁷ cells/0.1 ml PBMC cell suspension were fully mixed in a volume ratio of 1:1 and inoculated subcutaneously into NOD/SCID mice aged 5 to 7 weeks weighing 18 to 22 g, 0.2 mL per mouse. According to the body weight, the animals were randomly divided into three groups: Model group (PBS), 19G294 group (30 μg per mouse once daily), 18G272 group (30 μg per mouse once daily), with 6 animals in each group. Intravenous treatment with humanized bispecific antibodies (18G272 and 19G294) or the vehicle (PBS) started on the day of inoculation, continuously for 5 days. Two days after drug withdrawal, a second course of treatment was performed. The first treatment was 1 h after inoculation, that is, the first courses were D1, D2, D3, D4, D5. The second courses were D8, D9, D10, D11 and D12. The frequency of treatment was once a day.

Tumor volume: Tumor volume was measured twice or thrice a week. The length and width of the tumor were measured by vernier caliper. Tumor volume: V=(length×width²)/2. Tumor Growth Inhibition value: TGI (%)=(1−T/C)×100%. It was generally accepted that T represented the relative tumor volume (the ratio of the tumor volume at the time of measurement to the tumor volume at the time of grouping) in the administration group and C represented the relative tumor volume (the ratio of the tumor volume at the time of measurement to the tumor volume at the time of grouping) in the model group. However, mice in this trial were divided into groups according to the body weight after inoculation, and the T and C represented the actual tumor volume of the treatment group and control group.

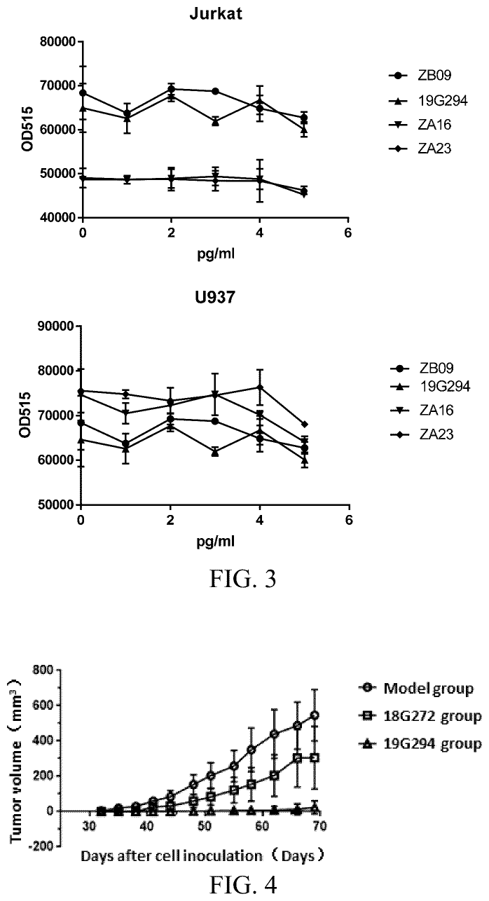

Tumor weight: Mice were euthanized at the end of the experiment, the tumor mass was stripped, rinsed with normal saline, blotted dry with filter paper, weighed and photographed. Tumor Growth Inhibition value: TGI (%)−(1−T_{TW}/C_{TW})×100%, T_{TW} represented average tumor weight of treatment group at the end of the experiment, C_{TW} represented average tumor weight of control group at the end of the experiment. 25 Conclusion: 18G272 can inhibit the tumor growth of mice bearing PC-3 (human prostate cancer) at the dose of 30 μg per mouse, and 19G294 can almost completely inhibit the tumor growth of mice bearing PC-3. These results were shown in FIG. 4 and Table 10.

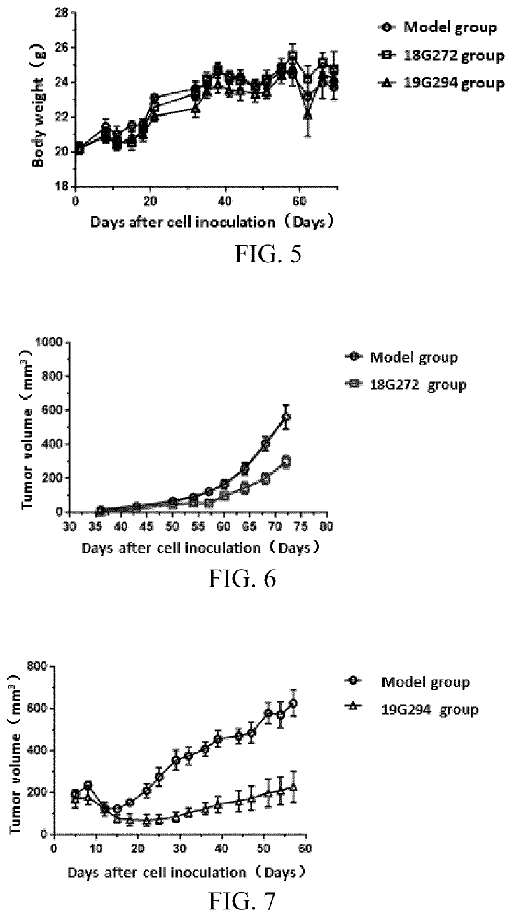

At the dose of 30 μg/mouse, the body weight of mice showed an upward trend in 19G294 and 18G272 groups, which was similar to that in the model group (control group), and the animals were well tolerated during the treatment. The results were shown in FIG. 5 and Table 10.

TABLE 10
Tumor growth and body weight of mice subcutaneously bearing PC-3

| | | Tumor weight on | Body weight on | Body weight on |
|---|---|---|---|---|
| | Tumor volume | D 69 | D 1 | D 69 |
| Groups | on D 69(mm³) | (mg) | (g) | (g) |
| Model group | 546.05 ± 146.04 | 473.88 ± 127.65 | 20.23 ± 0.37 | 23.75 ± 0.72 |
| 18G272 group | 305.82 ± 177.85 | 225.78 ± 120.25 | 20.22 ± 0.34 | 24.77 ± 1.01 |
| 19G294 group | 23.69 ± 15.13** | 11.28 ± 7.63** | 20.20 ± 0.28 | 24.27 ± 0.56 |

Note:
*P < 0.05, vs Model group;
**P < 0.01, vs Model group.

### IV Efficacy of Humanized Bispecific Antibody 18G272 in NOD/SCID Mice Subcutaneously Bearing NCI-H596 Human Lung Cancer Cell Xenografts

Methods: 8×10⁶ cells/0.1 ml NCI-H596 cell suspension and 1.6×10⁷ cells/0.1 ml PBMC cell suspension were fully mixed in a volume ratio of 1:1 and inoculated subcutaneously into NOD/SCID mice aged 5 to 7 weeks weighing 18 to 22 g, 0.2 mL per mouse. According to the body weight, the animals were randomly divided into two groups: Model group (PBS, 6 mice/group) and 18G272 group (30 μg per mouse once daily, 6 mice/group).

Administration route and administration frequency were the same as above.

General clinical observation, body weight, and tumor volume were measured as above.

Conclusion: 18G272 can significantly inhibit the tumor growth of mice bearing NCI-H596 (human lung adenosquamous carcinoma) at the dose of 30 μg per mouse. Body weight of mice in 18G272 group had an upward trend and animals were well tolerated during treatment at the dose of 30 μg per mouse. The results were shown in FIG. 6 and Table 11.

TABLE 11
Tumor growth and body weight of mice
subcutaneously bearing NCI-H596

| | Tumor volume | Body weight | Body weight |
|---|---|---|---|
| Groups | on D 72(mm³) | on D 1(g) | on D 72(g) |
| Model group | 560.84 ± 70.19 | 19.22 ± 0.54 | 23.95 ± 0.74 |
| 18G272 group | 298.46 ± 34.56* | 19.14 ± 0.33 | 24.52 ± 0.48 |

Note:
*P < 0.05, vs Model group.

### V Efficacy of Humanized Bispecific Antibody 19G294 in NOD/SCID Mice Subcutaneously Bearing NCI-H226 Human Mesothelioma Cell Xenografts

Methods: 5×10⁶ cells/0.1 ml NCI-H226 cell suspension and 1.5×10⁷ cells/0.1 ml PBMC cell suspension were fully mixed in a volume ratio of 1:1 and inoculated subcutaneously into NOD/SCID mice aged 5 to 7 weeks weighing 18 to 22 g, 0.2 mL per mouse. The animals were randomly divided into two groups according to body weight: Model group (PBS, 5 mice/group) and 19G294 group (60 μg per mouse once daily, 5 mice/group). Administration route and administration frequency were the same as above.

General clinical observation, body weight, and tumor volume were conducted as above.

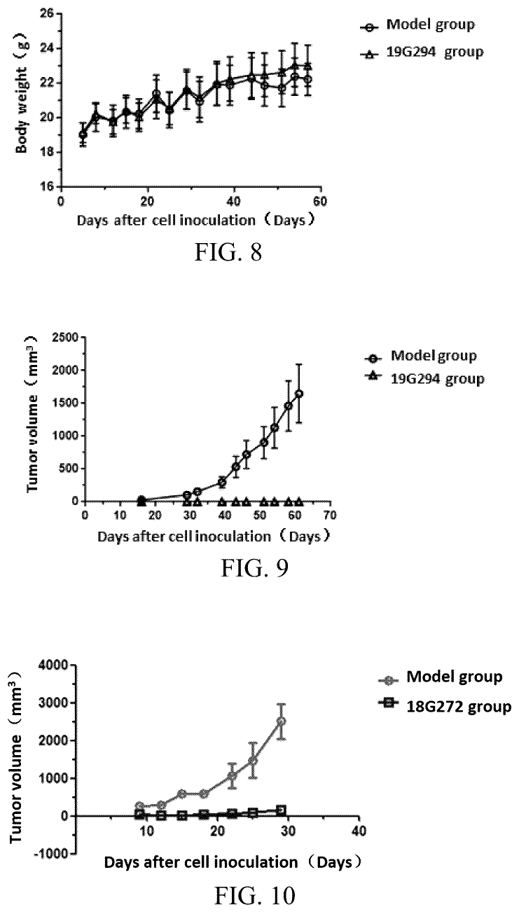

Conclusion: 19G294 can significantly inhibit the tumor growth of mice bearing NCI-H226 human lung adenosquamous carcinoma (mesothelioma) at the dose of 60 μg per mouse, as shown in FIG. 7. At the dose of 60 μg per mouse, body weight of mice in 19G294 group had an upward trend and animals were well tolerated during treatment. The results were shown in FIG. 8 and Table 12.

TABLE 12
Tumor growth and body weight of mice subcutaneously bearing NCI-H226

| | | | | | Body |
|---|---|---|---|---|---|
| | Tumor volume | Tumor weight | | Body weight | weight |
| Groups | on D 57(mm³) | on D 57(mg) | TGI ^{a}(%) | on D 1(g) | on D 57(g) |
| Model | 653.13 ± 74.92 | 372.58 ± 56.92 | — | 19.06 ± 0.67 | 22.22 ± 0.92 |
| group | | | | | |
| 19G294 | 228.60 ± 74.30** | 127.18 ± 47.60** | 66% | 19.28 ± 0.60 | 23.00 ± 1.19 |
| group | | | | | |

Note:
*P < 0.05, vs Model group.
^{a} indicated that TGI was calculated according to tumor weight on day 57 after cell inoculation.

### VI Efficacy of Humanized Bispecific Antibody 19G294 in NOD/SCID Mice Subcutaneously Bearing A498 Human Renal Cancer Cells Xenografts

Methods: 1×10⁷ cells/0.1 ml A498 cell suspension and 1×10⁷ cells/0.1 ml PBMC suspension were fully mixed in a volume ratio of 1:1 and inoculated subcutaneously into NOD/SCID mice aged 5 to 7 weeks weighing 18 to 22 g, 0.2 mL per mouse. According to the body weight, the animals were randomly divided into two groups: Model group (PBS) and 19G294 group (30 μg per mouse once daily), with 5 animals in each group.

Administration route and administration frequency were the same as above.

General clinical observation, body weight, tumor volume, and tumor weight were conducted as above.

Conclusion: 19G294 can completely inhibit the tumor growth of mice bearing A498 human renal cell carcinoma at the dose of 30 μg per mouse, as shown in FIG. 9 and Table 13. Mice in 19G294 group did not show weight loss and were well tolerated during treatment at the dose of 30 μg per mouse.

TABLE 13

| | Tumor volume | Tumor weight | | Body weight | Body weight |
|---|---|---|---|---|---|
| Groups | on D 61(mm³) | on D 61(mg) | TGI ^{a}(%) | on D 1(g) | on D 61(g) |
| Model | 1648.94 ± 444.85 | 1105.04 ± 342.68 | — | 20.68 ± 0.81 | 24.06 ± 0.81 |
| group | | | | | |
| 19G294 | 0.00 ± 0.00** | 0.00 ± 0.00** | 100% | 20.50 ± 0.50 | 24.18 ± 0.93 |
| group | | | | | |

Note:
*P < 0.05, vs Model group;
**P < 0.01, vs Model group.
^{a} indicated that TGI was calculated according to tumor weight on day 61 after cell inoculation.

### VII. Efficacy of Bispecific Antibodies 18G272 and 19G294 in NOD/SCID Mice Subcutaneously Bearing OS-RC-2 Human Renal Cancer Cells Xenografts

### Experiment 1

Methods: 3×10⁶ cells/0.1 ml OS-RC-2 cell suspension and 6×10⁶ cells/0.1 ml PBMC suspension were fully mixed in a volume ratio of 1:1 and inoculated subcutaneously into NOD/SCID mice aged 5 to 7 weeks weighing 18 to 22 g, 0.2 mL per mouse. The animals were randomly divided into two groups according to the body weight: Model group (PBS) and 18G272 group (30 μg per mouse once daily). There were six animals in each group.

Administration route and administration frequency were the same as above.

Conclusion: 18G272 can significantly inhibit the tumor growth of mice bearing OS-RC-2 at the dose of 30 μg per mouse, as shown in FIG. 10 and Table 14. Mice bearing OS-RC-2 were prone to die during the test. In this experiment, 18G272 can significantly improve the median survival time (MST) of the mice at a dose of 30 μg per mouse, reduce animal mortality, and mice in 18G272 group were well tolerated during the treatment and showed no weight loss. The results were shown in Table 14.

TABLE 14

| | | | | Body | Body | Survival |
|---|---|---|---|---|---|---|
| | Tumor volume | Tumor weight | TGI | weight | weight | rate |
| Groups | on D15(mm³) | on D29(mg) | ^{a}(%) | on D1(g) | on D15(g) | on D31 |
| Model | 598.55 ± 80.50 | 1656.15 ± 147.85 | — | 18.33 ± 0.18 | 18.07 ± 0.94 | 2/6 |
| group | | | | | | |
| 18G272 | 12.87 ± 12.87** | 96.80 ± 38.41 | 94.2% | 18.30 ± 0.19 | 19.43 ± 0.67 | 6/6 |
| group | | | | | | |

Note:
* P < 0.05, vs Model group;
**P < 0.01, vs Model group; After D15, the animals in each group began to die or were euthanized due to tumor growth.
^{a}indicated that TGI was calculated according to tumor weight on day 29 after cell inoculation.

### Experiment 2

Methods: 3×10⁶ cells/0.1 ml OS-RC-2 cell suspension and 6×10⁶ cells/0.1 ml PBMC suspension were fully mixed in a volume ratio of 1:1 and inoculated subcutaneously into NOD/SCID mice aged 5 to 7 weeks weighing 18 to 22 g, 0.2 mL per mouse. The animals were randomly divided into two groups according to the body weight: Model group (PBS) and 19G294 group (30 μg per mouse once daily). There were 5 animals in each group.

Administration route and administration frequency were the same as above.

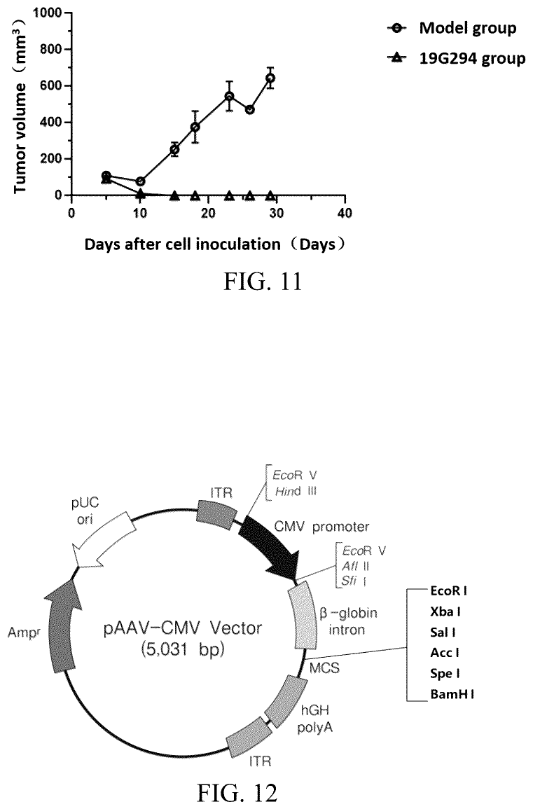

Conclusion: 19G294 can significantly inhibit the tumor growth of mice bearing OS-RC-2 at the dose of 30 μg per mouse, as shown in FIG. 11 and Table 15. Mice bearing OS-RC-2 were prone to die during the test. In this experiment, 19G294 can reduce the mortality of animals, prolong the survival time of animals, and mice in 196294 group were well tolerated during the treatment and showed no weight loss. The results were shown in Table 15.

TABLE 15
Tumor growth, body weight, and survival rate
of mice subcutaneously bearing OS-RC-2

| | | | Body | Body | Survival |
|---|---|---|---|---|---|
| | Tumor volume | Tumor volume | weight on | weight on | rate on |
| Groups | on D 23(mm³) | on D 29(mm³) | D 1(g) | D 23(g) | D 29 |
| Model | 545.91 ± 80.93 | 645.40 ± 56.68 | 19.04 ± 0.85 | 16.22 ± 1.49 | 2/5 |
| group | | | | | |
| 19G294 | 0.00 ± 0.00** | 0.00 ± 0.00** | 18.76 ± 0.57 | 20.34 ± 0.52* | 5/5 |
| group | | | | | |

Note:
*P < 0.05, vs Model group;
**P < 0.01, vs Model group;
Animals began to die or were euthanized after D 23

From the results of in vivo experiments, a variety of CD26 antibodies showed good therapeutic effects on a variety of different solid tumor cell models with no expression or low expression of CD45.

### Example 4 Drug Efficacy of rAAV Vector Containing Gene Encoding Anti-CD26 Antibody in Tumor-Bearing Mice

The above examples observed the killing effect of CD26 antibody on different types of cells in vitro and in vivo. To further observe the effect of gene therapy targeting CD26 on tumor cells, the following experiments were carried out:

### I. Construction of AAV Expressing Vector

A diagram of the AAV expressing vector backbone was shown in FIG. 12.

Construction of pAAV-GFP expressing vector:

GFP is green fluorescent protein. After amplification, GFP gene sequences (SEQ ID NO: 13) was cloned into multiple cloning site between XbaI and BamHI which is located between “beta introns enhanced sequence” and “polyA termination sequence” in vector pAAV-CMV, then pAAV-GFP expressing vector was formed as control.

Construction of pAAV-BiTE expressing vector:
The signal peptide gene (SEQ ID NO: 14) and BiTE gene (SEQ ID NO: 15) were fused, synthesized, amplified and cloned into multiple cloning site between BamHI and EcoRI which is located between “beta introns enhanced sequence” and “polyA termination sequence” in vector pAAV-CMV, then pAAV-21R23 expressing vector (containing CD26− CD3BiTE) was formed

### II. Production and Purification of Recombinant AAV Virus

293 T cell culture: After cell resuscitation, 293 T cells were cultured adherently in DMEM medium containing 10% FBS and 1% Glutamax. After passaged for 2-3 times, the cells with recovered condition can be used for packaging. The 150 mm cell culture dishes were coated with 15 ml of 0.1% gelatin for 30 min at room temperature. 293T cells were resuspended and counted after trypsin digestion. The coating solution in the culture dish was discarded. Each 150 mm dish was seeded with 1.2-1.8×10e7 293T cells, added with 30 ml 293T medium, and cultured overnight at 37° C. with 5% CO₂.

Transfection: The next morning, 293T cells were grown to 70-80% confluence and replaced with fresh 30 ml 293T medium for each dish, and replaced back into the incubator. Prepare transfection mixture, two AAV expressing vector plasmids prepared were added into serum-free DMEM medium with pRC6 and pHelper plasmid at a mass ratio of 1:1:1, respectively, each dish 30 μg total DNA. Then gently add Lipo8000 liposomes (Beyotime biotechnology, article number: C0533), gently mix and let stand for 30 min at room temperature. Add 1.5 ml transfection mixture to each dish, gentle blend, back into the incubator. After transfection for 24 hours, the medium was replaced with 30 ml of AAV harvest medium (DMEM medium containing 2% FBS, 1% Glutamax, 1% 1M HEPES) and returned to the incubator.

rAAV collection and purification: 72 h after infection, add 1/80 of culture volume of the 0.5 M EDTA (pH8.0) to the dish containing 293 T cells. The cells were resuspended, and collected in 50 ml sterile centrifuge tube. Centrifuge at 4° C. 2000 g for 10 minutes, discard supernatant, and collect cells precipitation, which was the host of 293 T cells containing AAV particles. AAVpro® Purification Kit Maxi (TAKARA product number: 6666) was used for purification and concentration of AAV, then AAV was packaged and stored at −80° C.

### III. Efficacy of Recombinant AAV Delivering BiTE in NOD/SCID Mice Bearing OS-RC-2 (Renal Cancer) Xenograft

Methods: 4×10⁷ cells/ml OS-RC-2 cell suspension and 6×10⁷ cells/ml PBMC suspension were fully mixed in a volume ratio of 1:1 and inoculated subcutaneously into NOD/SCID mice aged 5 to 7 weeks weighing 18 to 22 g. Purified GFP and 21R23 AAV samples were diluted to 1×10¹¹ vg/ml. OS-RC-2+PBMC cell suspension (0.1 ml) was mixed with PBS or AAV purified samples (0.1 ml) and injected subcutaneously into NOD-SCID mice, 0.2 ml for each mouse. There were Model group, group A (GFP) and group B (21R23), with 6 animals in each group. Tumor volume and weight was measured, mice survival state was observed from the 5th day of modeling.

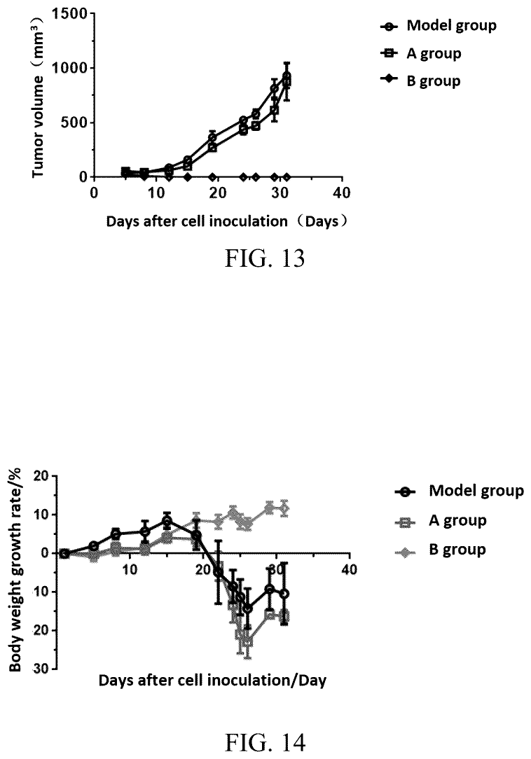

Conclusions: In this study, NOD-SCID mice were subcutaneously injected with the mixture of OS-RC-2 and PBMC cells to establish a human renal cell carcinoma xenograft model. A 10e10vg injection of recombinant AAV expressing BiTE was administered to each mouse. From the 8th day of modeling, the tumors in the model group and the GFP-A group began to increase significantly, and the tumor volume in the group B with AAV treatment was significantly inhibited. On the 12th day, the tumors in the group B completely regressed. The results were shown in FIG. 13 and Table 16.

TABLE 16

| | | Tumor volumn | | | | | | | | |
|---|---|---|---|---|---|---|---|---|---|---|
| Groups | n | D5 | D8 | D12 | D15 | D19 | D24 | D26 | D29 | D31 |
| Model | 6 | 39.83 ± | 42.90 ± | 87.41 ± | 160.68 ± | 364.68 ± | 524.87 ± | 579.71 ± | 814.84 ± | 931.78 ± |
| | | 10.33 | 2.07 | 10.59 | 26.64 | 65.20 | 27.57 | 36.85 | 66.66 | 87.99 |
| GFP | 6 | 55.71 ± | 45.46 ± | 63.47 ± | 100.32 ± | 268.65 ± | 435.40 ± | 470.98 ± | 613.31 ± | 877.31 ± |
| | | 5.47 | 4.88 | 12.67 | 12.18 | 31.37 | 48.23 | 38.69 | 63.52 | 110.40 |
| 21R23 | 6 | 28.31 ± | 7.93 ± | 0.00 ± | 0.00 ± | 0.00 ± | 0.00 ± | 0.00 ± | 0.00 ± | 0.00 ± |
| | | 6.01 | 1.63** | 0.00** | 0.00** | 0.00** | 0.00** | 0.00** | 0.00** | 0.00** |

**P < 0.01, vs Model;
* P < 0.05, vs Model

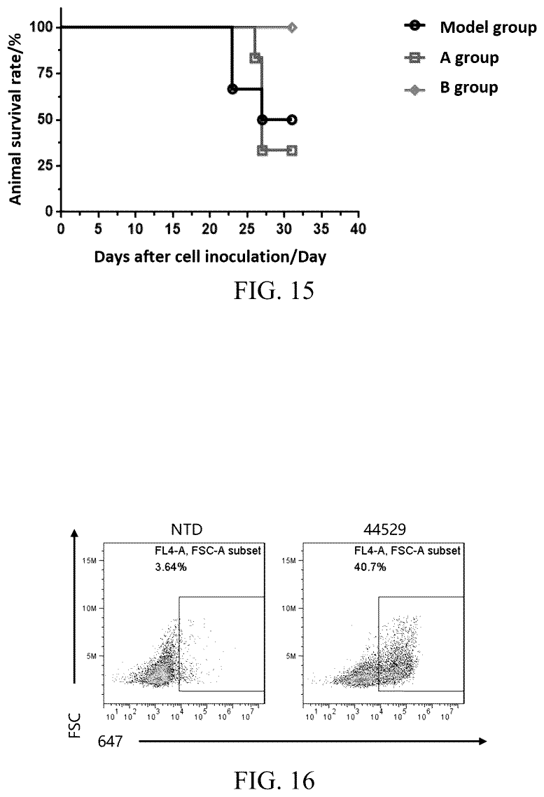

In addition, the OS-RC-2 model can lead to animal death during the test, and animals in AAV treatment group had no weight loss, and were well tolerated during the treatment, as shown in FIGS. 14 and 15.

In conclusion, the recombinant AAV delivering BiTE had a significant therapeutic effect on CD26-expressing renal cancer cells with no or low CD45 expression after a single dose of administration.

### Example 5 Evaluation of Cytotoxicity of CD26-Targeting CAR-T Cells on CD26−CD45−, CD26+CD45−, and CD26+CD45+ Cells

In order to further observe the CD26-targeting CAR-T cells immunotherapy on tumor cells and the relevance of CD45 expression, carry out the following tests:

### 1. CAR-T Cell Construction

Recombinant CAR gene contains BamHI restriction enzyme cutting site, Kozak sequence, CD8α signal peptide, anti-CD26 single chain antibody fragment shown as VL-Linker-VH, CD8α hinge, CD8α transmembrane domain, CD28 costimulatory domain, CD3zeta intracellular domain, termination codon, SalI restriction enzyme cutting site. The full sequence of the recombinant CAR gene was synthesized (SEQ ID NO: 3) and inserted into the lentiviral vector pWPT-GFP by BamHI and SalI double digestion to replace the GFP gene to form the lentiviral expressing vector pWPT-44529.

The constructed PWPT-44529 plasmid was packaged with lentivirus and purified, and then the recombinant CAR lentivirus was used to infect healthy donor T cells. IL2 and activator were used to stimulate the proliferation of CAR-T cells. After proliferation, the positive rate of CAR was detected by flow cytometry. Positive rate of lentivirus-infected T cells reached 40.7%.

### 2. Efficacy Evaluation of CAR-T Cells

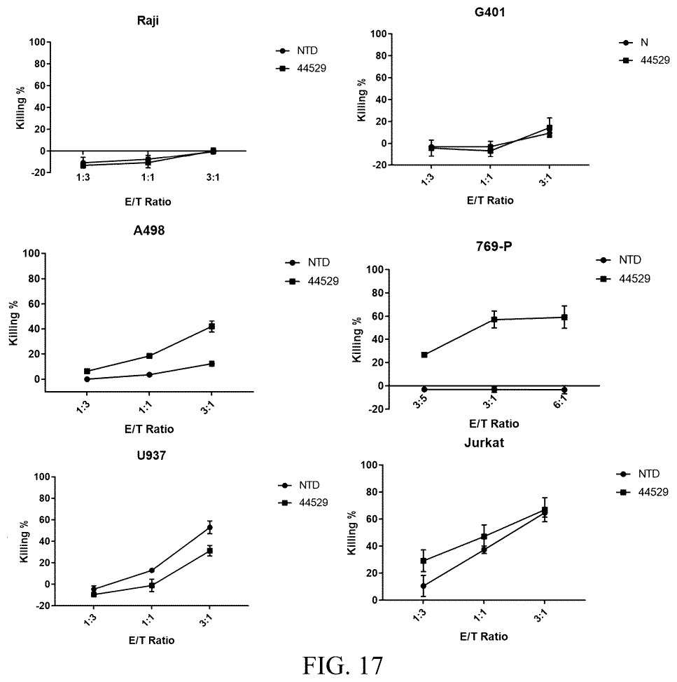

The transfected CAR-T cells (44529) and untransfected T cells (NTD) were co-cultured with different target cells in 96-well plates. The target cells included: CD26+CD45− A498 and 769-P cells, CD26− G401 and Raji cells, CD26+CD45+ U937 and Jurkat cells. After 24 hours of co-culture, the supernatant in a 96-well plate was taken. The DOJINDO LDH detection kit (CK12) was used to detect LDH release of target cells in the supernatant to calculate the killing rate of CAR-T cells and NTD cells with different effector-target ratios, as shown in FIG. 17.

The results showed that CD26-targeting CAR-T cells had no specific killing effect on CD26 negative target cells Raji and G401, but on CD26+CD45− cells (A498, 769-P), CD26 CAR-T cells showed significantly higher killing effect than the control T cells, proving CD26 CAR-T target specificity. But for CD26+CD45+ double positive cells (U937, Jurkat), CD26 CAR-T effect was significantly inhibited, and no specific killing was detected.

In summary, anti-CD26 antibody and other gene therapy or cell therapy, such as CD26-targeting AAV gene therapy and CD26-targeting CAR-T cell therapy, had significant killing activity against CD45 negative or low expression tumor cells in vitro and in vivo. And CD45 high expression tumor cells had no significant damage.

Based on the above research results, this application provides the use of CD45 in screening the effectiveness of CD26 antibody or derivative thereof, which can effectively improve the accuracy and effectiveness of CD26 antibody or derivative thereof in tumor immunotherapy, and improve the clinical benefit of patients. Novel mechanism for the effectiveness and accuracy of CD26 therapy as new target for cancer immunotherapy has been identified in this application.

1-10. (canceled)

11. A method of using a CD26 antibody or a derivative thereof to treat a tumor with CD26 expression but without CD45 expression, or a tumor with CD26 expression and with low CD45 expression, wherein the tumor is a solid tumor or a hematological tumor.

12. The method of claim 11, wherein the tumor is a solid tumor.

13. The method of claim 11, wherein the tumor is selected from the group consisting of: a renal cancer, a lung cancer, and a prostate cancer.

14. The method of claim 13, wherein the lung cancer comprise a lung squamous carcinoma.

15. The method of claim 11, the CD26 antibody is a full-length antibody, a Fab fragment, a FV fragment, a linear antibody, a single-chain antibody, a nanobody, a bispecific antibody, or a multi-specific antibody that specifically binds to CD26.

16. The method of claim 11, the derivative of CD26 antibody is a Car-T targeting CD26.

17. The method of claim 11, the derivative of CD26 antibody is an rAAV vector comprising a gene encoding anti-CD26 antibody.

18. A method for screening the effectiveness of treatment with CD26 antibody or derivative thereof to a subject, which comprises a step of detecting the expression of CD45 in lesions; when CD45 has a low expression, the treatment with CD26 antibody or derivative thereof to the subject is effective or has a high probability of effectiveness; when CD45 has a high expression, the treatment with CD26 antibody or derivative thereof to the subject is ineffective or has a high probability of ineffectiveness.

19. The method of claim 18, wherein a CD45 antigen or an anti-CD45 antibody is used to screen the effectiveness of treatment with CD26 antibody or derivative thereof to the subject.

20. The method of claim 18, wherein the subject has a tumor, and the tumor is a solid tumor or a hematological tumor.

21. The method of claim 20, wherein the tumor is selected from the group consisting of: a renal cancer, a lung cancer, and a prostate cancer.

22. The method of claim 21, wherein the lung cancer comprise a lung squamous carcinoma.

23. A method for screening the effectiveness of treatment with CD26 antibody or derivative thereof to a subject, which comprises a step of detecting the expression of CD45 in lesions; when CD45 has no expression, the treatment with CD26 antibody or derivative thereof to the subject is effective or has a high probability of effectiveness; when CD45 has an expression, the treatment with CD26 antibody or derivative thereof to the subject is ineffective or has a high probability of ineffectiveness.

24. The method of claim 23, wherein a CD45 antigen or an anti-CD45 antibody is used to screen the effectiveness of treatment with CD26 antibody or derivative thereof to the subject.

25. The method of claim 23, wherein the subject has a tumor, and the tumor is a solid tumor or a hematological tumor.

26. The method of claim 25, wherein the tumor is selected from the group consisting of: a renal cancer, a lung cancer, and a prostate cancer.

27. The method of claim 26, wherein the lung cancer comprise a lung squamous carcinoma.


SEQUENCE LISTING (15 sequences)

SEQ ID NO: 1 - protein, 492 aa, synthetic construct
DIVMTQSASSLPVTPGEPASISCRSDKSLLHSNGNTYLYWFLQKPGQSPQVLIYRMSNLASGVPDRFSGSGSGTDFTLTISRVEAEDVGVYYCFQQLEYPYTFGQGTKLEIKGGGGSGGGGSGGGGSQVQLVQSGAEVVKPGASVKISCKASGFNIKDTYIYWVRQRTGQGLEWIGRIEPMNGNTKYDPKFQGRLTMTANTSKNTAYLQLSSLTSEDTAVYYCSRGGWLLLYVMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 2 - protein, 492 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIKGGGGSGGGGSGGGGSEVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 3 - DNA, 1495 bp, synthetic construct
  source 1..1495: mol_type = other DNA
ggatccgccgccaccatggccctgcctgtgaccgccctgctgctgccactggccctgctgctccacgccgctagacctgatatcctgctgacacagagccccagctccctgagcgccacacctggcgagagggccaccatcacatgtagagccagccagggcatcaggaacaatctgaattggtaccagcagaagcccggccaggcccctagactgctgatctactactccagcaacctgcaaagcggcgtgccttccagattttccggctccggcagcggcaccgactttacactgacaatctccagactgcaacccgaggatgtggccgcctactactgccagcagagcatcaagctgcccttcacattcggctccggcacaaaggtggagatcaagggcggcggcggcagcggaggaggaggaagcggaggaggcggcagcgaggtgcagctggtggagagcggcgccggagtgaagcagcccggaggaaccctgagactgacctgtacagccagcggcttctccctgacaacctacggcgtgcactgggtgaggcaggcccccggaaagggcctggagtgggtgggagtgatctggggcgatggcagaaccgattacgacgccgcctttatgtccagagtgacaatcagcaaggacacatccaagtccaccgtgtacctgcagatgaactccctgagggccgaggacacagccgtgtactactgcatgagaaacaggcacgattggttcgactactggggccagggcacaacagtgaccgtgtcctccacaacaacccccgcccccaggcctcctacacccgctcctacaatcgccagccagcccctgagcctgaggcctgaggcctgccgacctgccgctggaggagctgtgcacaccaggggcctggattttgcctgtgatatctacatctgggcccctctggccggcacctgtggcgttctgctgctgtccctggtcattaccctgtactgtaggagcaagagaagcaggctgctgcactccgactacatgaatatgacacctagaaggcctggccccaccaggaagcactaccagccctacgcccctcccagggacttcgccgcttacagaagcagggtgaagttctccaggtccgccgacgcccccgcctaccaacagggacagaatcagctgtacaacgagctgaacctgggcagaagagaggagtacgacgtgctggataagaggagaggcagggaccccgagatgggcggcaagccacagaggagaaagaacccccaggagggcctgtacaacgaactgcaaaaggataagatggccgaggcctactccgagatcggcatgaagggcgagagaagaagaggcaagggccacgacggcctgtaccagggcctgtccaccgccaccaaggatacctacgatgccctgcacatgcaggccctgccccccagataatgagtcgacgtcggccatagcggccgcggaa

SEQ ID NO: 4 - protein, 1020 aa, synthetic construct
DIVMTQSASSLPVTPGEPASISCRSDKSLLHSNGNTYLYWFLQKPGQSPQVLIYRMSNLASGVPDRFSGSGSGTDFTLTISRVEAEDVGVYYCFQQLEYPYTFGQGTKLEIKGGGGSGGGGSGGGGSQVQLVQSGAEVVKPGASVKISCKASGFNIKDTYIYWVRQRTGQGLEWIGRIEPMNGNTKYDPKFQGRLTMTANTSKNTAYLQLSSLTSEDTAVYYCSRGGWLLLYVMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGGGGSGGGGSGGGGSDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSGGGGSGGGGSGGGGSGGGGSGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 450 aa, synthetic construct
EVQLVQSGAELVKPGASVKLSCAASGFNIKDTYIYWVRQAPGQGLEWVGRIEPMNGNTKYDPKFQGRFTMTANTSKNTAYLQLSSLRSEDTAVYYCSRGGWLLLYVMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 219 aa, synthetic construct
DIVMTQSPSSLSVTPGERVSISCRSDKSLLHSNGNTYLYWFLQKPGQSPQVLIYRMSNLASGVPDRFSGSGSGTDFTLTISRVEAEDVGVYYCFQQLEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 449 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 8 - protein, 213 aa, synthetic construct
DIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKRTVAAPSVFICPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 9 - protein, 450 aa, synthetic construct
QVQLVQSGAEVVKPGASVKISCKASGFNIKDTYIYWVRQRTGQGLEWIGRIEPMNGNTKYDPKFQGRLTMTANTSKNTAYLQLSSLTSEDTAVYYCSRGGWLLLYVMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
DIVMTQSASSLPVTPGEPASISCRSDKSLLHSNGNTYLYWFLQKPGQSPQVLIYRMSNLASGVPDRFSGSGSGTDFTLTISRVEAEDVGVYYCFQQLEYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 449 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHTCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 12 - protein, 213 aa, synthetic construct; identical to SEQ ID NO: 8

SEQ ID NO: 13 - DNA, 738 bp, synthetic construct
  source 1..738: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcgatag

SEQ ID NO: 14 - DNA, 63 bp, synthetic construct
  source 1..63: mol_type = other DNA
atggagcactccaccttcctgagcggcctggtgctggccacactgctgtctcaggtgtccccc

SEQ ID NO: 15 - DNA, 1479 bp, synthetic construct
  source 1..1479: mol_type = other DNA
gacatcgtgatgacccagagcccatccagcctgtctgtgacaccaggagagagggtgtccatcagctgccggagcgataagtctctgctgcattctaacggcaatacctacctgtattggtttctgcagaagcccggccagtcccctcaggtgctgatctacagaatgtctaacctggcctccggagtgccagaccgcttctctggatccggaagcggcacagacttcaccctgacaatctccagggtggaggctgaggacgtgggcgtgtactattgcttccagcagctggagtacccctatacctttggccagggcacaaagctggagatcaagggaggaggaggatccggaggaggaggaagcggcggcggcggctctgaggtgcagctggtgcagtccggagctgagctggtgaagccaggagctagcgtgaagctgtcttgtgccgcttccggcttcaatatcaaggacacctacatctattgggtgagacaggctcctggacagggcctggagtgggtgggccgcatcgagcctatgaacggcaatacaaagtacgatccaaagttccagggcaggtttaccatgacagccaacaccagcaagaatacagcttatctgcagctgtcttccctgaggtccgaggacaccgccgtgtactattgtagccggggaggatggctgctgctgtacgtgatggattattggggccagggcaccctggtgacagtgagctctggaggaggaggatctgatgtgcagctggtgcagagcggagctgaggtgaagaagccaggagcttctgtgaaggtgtcctgtaaggccagcggctacaccttcacaagatataccatgcactgggtgcgccaggctccaggacagggactggagtggatcggctacatcaacccctctagaggctacaccaattatgccgactccgtgaagggccgctttacaatcaccacagataagagcacctctacagcttacatggagctgtccagcctgaggtccgaggacaccgccacatactattgcgctcggtactatgacgatcattactgtctggattactggggtcagggcaccacagtgaccgtgtcttctggcggcggcggcagtggcggcggcggctccggcggcggcggctccgacatcgtgctgacccagtcccctgccacactgtccctgagcccaggagagagggccaccctgagctgccgggcttctcagtccgtgagctacatgaactggtatcagcagaagccaggcaaggctcccaagagatggatctacgatacatctaaggtggcctccggagtgccagctcgcttctctggctctggaagcggaaccgactatagcctgacaatcaactctctggaggccgaggatgccgctacctactattgtcagcagtggagctctaatcccctgacctttggcggcggcacaaaggtggagatcaagtga